(19) United States
(12) Patent Application Publication
(10) Pub. No.: US 2018/0280234 A1
(43) Pub. Date: Oct. 4, 2018

(54) ENTERAL FEEDING DEVICE

(71) Applicant: Pronova Biopharma Norge AS, Lysaker (NO)

(72) Inventors: Merethe H Brevik-Andersen, Oslo (NO); Emilie Lasson, Oslo (NO); Hilde Nyheim, Jar (NO); Marianne Weiby Wulff, Oslo (NO)

(21) Appl. No.: 15/545,660

(22) PCT Filed: Jan. 27, 2016
(86) PCT No.: PCT/EP2016/051690

§ 371 Date: Jul. 21, 2017

(30) Foreign Application Priority Data
Jan. 30, 2015 (NO) 150142

(51) Int. Cl. A61J 1/10; B65D 35/28; B65D 81/32; A61J 1/14; A61J 9/00; A61J 15/00; A61K 9/00; A61K 31/202; A23L 33/12

(52) U.S. Cl. CPC A61J 1/10; B65D 35/28; B65D 81/3266; A61J 1/1412; A61J 1/1481; A23V 2002/00; A61J 15/0003; A61J 15/0069; A61K 9/0053; A61K 31/202; A23L 33/12; A61J 9/005

(57) ABSTRACT

The present invention relates to a fluid dispensing container in the field of enteral nutritional feeding, and in. particular to a dispenser for dispensing of a supplemental enteral fluid to a subject. Particularly, the invention provides a small-volume dispenser p refilled with an enteral fluid, which can be coupled with a feeding or medical tube port. Preferably, the enteral fluid is a composition of omega-3 fatty acids. The invention further provides a method for administering an enteral fluid to a subject using such dispenser. Yet further, the invention provides a method for supplementing an enteral nutritional product with an enteral fluid using such dispenser.

### FIELD OF THE INVENTION

The present invention relates to a fluid dispensing container in the field of enteral nutritional feeding, and in particular to a container for dispensing of a supplemental enteral nutrient or nutrients, herein called enteral fluid, to a subject. Particularly, the invention provides a small-volume dispenser prefilled with an enteral fluid which can be coupled with a feeding or medical tube port. The invention further provides a method for administering an enteral fluid to a subject using such dispenser. Yet further, the invention provides a method for supplementing an enteral nutritional product with an enteral fluid using such dispenser.

### BACKGROUND OF THE INVENTION

Due to a variety of reasons patients may not be able to obtain the necessary nutrition by ingesting food through the mouth, i.e. orally. Therefore, it has been known to provide medical enteral nutrition by nutritional supplements such as by tube feeding. Tube feeding is given to provide nutrition to patients who cannot obtain nutrition by swallowing, using a device such as a nasogastric feeding tube or a nasojejunal feeding tube, or by using a percutaneous endoscopic gastrostomy (PEG) or PEG-jejuno-feeding system. Being fed by a feeding tube is called enteral feeding, comprising all of the abovementioned tube feeding modes, and the nutrition used in such feeding is called enteral nutrition.

The feeding of a liquid enteral nutritional product from a hangable container is well known, such as from a bottle or a plastic bag with a bottom outlet connecting to a drip chamber and the latter to a flexible tubing, or lumen, leading to a nasogastric tube or a feeding tube inserted through a gastrostomy or a jejunostomy, by gravity flow or aided by a pump. Enteral nutrition may be supplied in a pre-filled, ready-to-hang container, or placed in such a container by a caregiver, or may be supplied by a syringe or cartridge. However, the selection of diets, particularly special diets is limited. This narrows the choices of diet modifications that might significantly benefit the patient. In addition, a number of nutrients as well as pharmaceuticals and other ingredients are not stable during heat sterilization or during storage unless proper precautions are taken, or may not be mutually compatible with other desired ingredients or package material for an extended period of time. Thus, there are nutrients and pharmaceuticals that may be desirable to enterally administer to a patient which are not readily prepared and formulated or packed together with other enteral nutrients. Accordingly, for some nutrients or pharmaceuticals there is a need for individually packed supplemental products that may be supplied to a subject enterally independent of the enteral nutrition normally given.

WO96/21481 discloses an apparatus for adding ingredients to a liquid enteral nutritional product during delivery of the nutritional product from a supply thereof, such as a hangable container, to a feeding tube delivering the nutritional product to the gastrointestinal tract of a patient. The apparatus comprises: a formulation chamber, connectable to a supply container of a liquid enteral nutritional product, the formulation chamber having an inlet and an outlet, at least one beneficial agent in controlled release dosage form, each controlled release dosage form unit being disposed in the formulation chamber so as to be wetted by or immersed in the liquid enteral nutritional product traversing the formulation chamber, and each beneficial agent being dispersible in the medium of the liquid enteral nutritional product, and fluid communication means connecting the outlet of the formulation chamber to a tube for feeding the modified enteral nutritional product, containing the so-added at least one beneficial agent, into the gastrointestinal tract of a patient. Further, the invention disclosed provides a method for modifying a liquid enteral nutritional product using such apparatus. The disclosed apparatus is rather complicated and a drawback is that the beneficial agent cannot be administered independently of the nutritional main product as the formulation chamber comprising the beneficial agent is coupled directly to the supply container for the liquid enteral nutritional product, and the nutritional product traverses the formulation chamber containing the beneficial agent. US2008/0319391 is directed towards a self-powered, enteral tube feeding device. The device includes a fluid container and a removable lid for pressure sealing the container. The container has a size big enough to hold one to three feedings and a preferred size for the container is 28 oz. Hence, the device is for administration of one of more meals and is re-usable. U.S. Pat. No. 5,372,578 is directed to a liquid delivery apparatus including a stored energy source disposed within an aseptic dispenser for delivering a nutritional liquid at a controlled uniform rate. The apparatus comprises a housing, a chamber and a liquid passageway having an inlet and an outlet. The device further include means for venting. These devices are intended for bigger volumes such as for full meals, and they include features to control rate of administration, avoidance of backflow etc. However, these disclosures do not provide devices or methods for easy delivery of smaller volumes of e.g. supplemental enteral fluids to a subject via a tube port.

An enteral nutritional product is usually given to a subject using a feeding tube. A single port tube may be used when the tube feeding will be given from a bag. It can be run through a pump that will deliver a precise amount of feeding at a given time. Alternatively, a tube with two or more ports may be used, typically having a larger port for the feed and a smaller port for medicines. The medical port is typically used for adding medications using a syringe, but it has also been experienced that nutritional fluids in smaller volumes are administered to a subject using a syringe and giving such through either of the ports. Drawbacks with using a syringe is e.g. the risk of spilling and the difficulties with handling a syringe. Further, all relevant nutritional fluids are not available in a syringe and would have to be transferred from other packages, into the syringe prior to attaching this to the port, using many operating steps. Such steps may also defer the quality of the nutritional fluid product. For supplement of fatty acids such as omega-3 fatty acids as enteral nutrition some healthcare personnel have explained that such oils have been pulled out of capsules by syringes for transfer to a tube port. Further, not all relevant nutrients and pharmaceuticals are suitable for packing in prefilled syringes, e.g. due to stability issues. For the reasons stated above, there is a need for a feasible method and for a dispenser for administering supplemental enteral fluids to a subject, wherein no syringe is used and wherein the supplemental enteral fluid can be administered to a patient independently of the normal enteral nutrition provided to the subject, with no requirement for reconstitution and with no use of a pump.

### BRIEF SUMMARY OF THE INVENTION

With the dispenser of the invention, the problem is solved by providing a small-volume dispenser for one time use, prefilled with an enteral fluid. The dispenser can be coupled with a tube port, it is easy to handle and to dispense and it provides protection to the enteral fluid in the dispenser. The dispenser is disposable. The dispenser provided is an alternative to syringes and has improved functionality, is more user-friendly, reliable and/or secure compared to such. As the features and small volume of the dispenser make it designed for being handheld the invention provides a feasible device and method for easily administering a small volume of supplemental enteral fluid to a subject. Further, the dispenser can be commercially produced at low cost and also allows for discard after use.

Hence, in a first aspect the invention provides a handheld dispenser for an enteral fluid, wherein the dispenser comprises

i) a flexible body portion providing a reservoir for the enteral fluid, the flexible body portion comprising a wall material compatible with the enteral fluid, wherein the flexible body portion having arranged thereto;

ii) an outlet connectable with a feeding tube.

Accordingly, the dispenser of the invention is for use for an enteral fluid, or is useful for an enteral fluid.

### BRIEF DESCRIPTION OF THE DRAWINGS

FIG. 1 is related to Example 1 providing oxidation parameters for different ampoule materials filled with concentrated fish oil.

FIG. 2 depicts a potential wall material for the dispenser of the present invention consisting of a 3 layer structure, in addition to two adhesive layers.

FIGS. 3a-c depict alternative dispensers according to the present invention wherein the flexible body portion has a pouch format.

FIGS. 4a-c depict one alternative dispenser according to the present invention comprising further elements.

FIG. 5a depicts one alternative dispenser according to the present invention wherein the flexible body portion has a tube format. FIGS. 5b and c provide cross-sections of alternative dispensers.

FIG. 6a depicts a dispenser of the present invention coupled to the port of a tube. FIG. 6b depicts a pressure means which may be attached to the dispenser as shown in FIG. 6a.

FIG. 7 depicts one alternative dispenser according to the present invention wherein the flexible body portion comprises two compartments.

FIGS. 8a, b and c depict two alternative dispensers according to the present invention of the bellow-type. FIG. 8b is a side view of FIG. 8a.

FIGS. 9a and b depict two alternative configurations of the connection between the outlet of the dispenser of the present invention and a port.

FIGS. 10a-f depict alternative closure means for the outlet of the dispenser of the present invention.

### DETAILED DESCRIPTION OF THE PREFERRED EMBODIMENTS

Accordingly, in a first embodiment the dispenser of the invention is prefilled with an enteral fluid, wherein the enteral fluid is preferably a supplement to the standard enteral nutrition a subject is receiving. The dispenser is hence prefilled with the relevant supplemental enteral fluid, sealed and can be stored for later use. The dispensers can be easily connected to a feeding tube for administration to a subject when needed. The subject is typically an adult. Once the dispenser has been prepared, filled and sealed it is non-refillable. The dispenser of the invention is intended for administration of smaller volumes of enteral fluids including nutrients and pharmaceuticals, such as those which are not sufficiently provided in the available standard choices of diets, or which the subject has an increased need for e.g. due to a deficiency or distinct nutritional requirement, and which should be provided to the subject as a supplement. Accordingly, the dispenser is for delivery of an enteral fluid comprising nutrients and/or pharmaceuticals, which can be selected from the group including Enteral formulas for Special Medical use, Foods for Specified Health Uses, Food for Special Medical Purposes (FSMP), Food for Special Dietary Use (FSDU), medical nutrition, medical food, clinical nutrition, neutraceuticals and supplements. Accordingly, in one embodiment the dispenser of the invention is prefilled with an enteral fluid selected from the group above.

The dispenser is disposable, preferably for one time use, and provides a single dose of a nutrient or pharmaceutical. The total volume is e.g. of 1 to 30 ml, more preferably 2 to 20 ml, even more preferably 2 to 15 ml, and most preferably 2 to 8 ml. In a particularly preferred embodiment the dispenser has a total volume of about 3-6 ml. The hand-held dispenser is connectable with a feeding tube and is manually operated.

The enteral fluid filled in the dispenser is suitable as a tube feed, i.e. enteral nutrition, and can easily be administered by a feeding tube. In one embodiment, the dispenser is prefilled with an enteral fluid comprising a composition of fatty acids or lipids and derivatives thereof, essential nutrients or pharmaceuticals, or any combinations thereof. Hence, one aspect of the invention provides a dispenser according to the first aspect containing an enteral fluid comprising a composition of fatty acids or lipids and derivatives thereof, essential nutrients or pharmaceuticals, or any combinations thereof.

Particularly, the enteral fluid is contained in the reservoir of the dispenser. Preferably, the dispenser is filled with a composition comprising fatty acids or lipids. The group of lipids include sterols and sterol esters. In a preferred embodiment, the dispenser is prefilled with an enteral fluid comprising a composition which is a fatty acid oil mixture comprising long chain polyunsaturated (LC-PUFA) fatty acids, such as omega-3 fatty acids or omega-6 fatty acids. Particularly the PUFAs originate from marine oil, i.e. fish oil, but may also be derived from algae oil, plant based oil or microbial oil. The PUFAs may be in different forms such as free fatty acids, esters, e.g. C1-C4 alkyl esters, preferably ethyl esters, phospholipids, mono/di/tri-glycerides and salts thereof. All concentrations and ratios may be used. In a preferred embodiment, the enteral fluid comprises a fatty acid oil mixture of long chain polyunsaturated (LC-PUFA) omega-3 fatty acids. In a preferred embodiment, the fatty acid oil mixture comprises either eicosapentaenoic acid (EPA) or docosahexaenoic acid (DHA), or a mixture of such. In another embodiment, the enteral fluid comprises a fatty acid oil mixture of long chain polyunsaturated (LC-PUFA) omega-6 fatty acids. Particularly, such omega-6 fatty acids are selected from Linoleic acid (LA), Conjugated linoleic acid (CLA), Gamma-linolenic acid (CGLA), Dihomo-gamma-linolenic acid (DGLA), Docosapentaenoic acid (DPA) and Arachidonic acid (AA). In one embodiment, the enteral fluid comprises a fatty acid oil mixture comprising both omega-3 and omega-6 fatty acids.

In one embodiment, the enteral fluid comprises a fatty acid oil mixture comprising at least 25 weight % of EPA and/or DHA, more preferably at least 35%, 45%, 55%, 65%, 75%, 85% or 95% of EPA and/or DHA. Higher concentrations are preferred and in one embodiment, the fatty acid oil mixture comprises at least 55 weight % of at least one of EPA and DHA by the weight of the fatty acids therein. In one embodiment the fatty acid oil mixture of the enteral fluid comprises high concentrations of one fatty acid, preferably either EPA or DHA. In at least one embodiment, the fatty acid oil mixture comprises at least 75 percent EPA and DHA by weight of the fatty acid oil mixture, of which at least 95 percent is EPA. In another embodiment, the fatty acid oil mixture comprises at least 80 percent EPA and DHA by weight of the fatty acid oil mixture, of which at least 95 percent is EPA. In yet another embodiment, the fatty acid oil mixture comprises at least 90 percent EPA and DHA by weight of the fatty acid oil mixture, of which at least 95 percent is EPA.

In another embodiment, the fatty acid oil mixture comprises at least 75 percent EPA and DHA by weight of the fatty acid oil mixture, of which at least 95 percent is DHA. For example, in one embodiment, the fatty acid oil mixture comprises at least 80 percent EPA and DHA by weight of the fatty acid oil mixture, of which at least 95 percent is DHA. In another embodiment, the fatty acid oil mixture comprises at least 90 percent EPA and DHA by weight of the fatty acid oil mixture, of which at least 95 percent is DHA.

In another embodiment, the fatty acid oil mixture of the enteral fluid comprises mixtures of EPA and DHA. In some embodiments of the present application, the weight ratio of EPA:DHA of the fatty acid oil mixture ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to 2 about:1. In at least one embodiment, the weight ratio of EPA:DHA of the fatty acid oil mixture ranges from about 1:2 to about 2:1. In at least one embodiment, the weight ratio of EPA:DHA of the fatty acid oil mixture ranges from about 1:1 to about 2:1. In at least one embodiment, the weight ratio of EPA:DHA of the composition ranges from about 1.2 to about 1.3. Such fatty acid oil mixture concentrates include, but are not limited to, the following examples: about 360 mg EPA and 240 mg DHA pr g oil, 400 mg EPA and 200 mg DHA pr g oil, 500 mg EPA and 200 mg DHA pr g oil, 150 mg EPA and 500 mg DHA pr g oil, 460 mg EPA and 380 mg DHA pr g oil, above 900 mg EPA pr g oil, above 900 mg DHA pr g oil, and 97% EPA. The total doses are e.g. from 1-20 g in total of EPA and DHA. In addition, other omega-3 fatty acids than EPA and DHA may be present.

In another embodiment, the dispenser is prefilled with other fatty acids and lipids such as naturally fatty acids and structurally modified/enhanced fatty acids (SEFAs) of all lengths and degrees of unsaturation, in all forms including free fatty acids, alkyl esters, phospholipids and mono/di/tri-glycerides, and derivatives thereof. In one such embodiment, the enteral fluid comprises omega-6 fatty acids, such as a mixture of omega-6 and omega-3 fatty acids.

The compositions for the enteral fluid presently disclosed may further comprise at least one antioxidant. Examples of antioxidants suitable for the present disclosure include, but are not limited to, a-tocopherol (vitamin E), calcium disodium EDTA, alpha tocoferyl acetates, butylhydroxytoluenes (BHT), butylhydroxyanisoles (BHA), ascorbyl palmitate and carotenoids such as beta-carotene and astaxanthin.

In yet another embodiment, the dispenser is prefilled with essential nutrients e.g. vitamins, e.g. vitamin D or E, and minerals either alone or in combination with fatty acids or lipids, such as those described above; with pharmaceuticals such as pain killers; e.g. paracetamol/acetaminophen, ibuprofen, naproxen, acetylsalicylic acid; digestion aids such as lactulose; or anti emetics such as metochlopramide.

In yet another embodiment the dispenser is prefilled with fatty acids or lipids in combination with lipases for hydrolysis of the fatty acids or lipids. The lipases may be mixed with the fatty acid or lipid, or may be contained in a separate compartment of the dispenser, such as in a second compartment of the flexible body portion, e.g. as shown in FIG. 7.

Formulated forms of all the above are all encompassed by the definition of the enteral fluid, including emulsions. Such emulsion may include a protein or non-protein emulsifier, including surfactants. Examples of formulations for fatty acids and lipids are Self Micro Emulsifying Drug Delivery System (SMEDDS), Self Nano Emulsifying Drug Delivery Systems (SNEDDs) and Self-Emulsifying Drug Delivery Systems (SEDDs) which form an emulsion in an aqueous solution. Typically, such formulations provide a preconcentrate composition comprising a fatty acid oil mixture and at least one surfactant. Relevant formulations to use according to the invention are disclosed in WO2010/103404 of the applicant. For example, the enteral fluid may be in the form of a pre-concentrate of any of the above fatty acids and a surfactant, such as a polysorbate, which spontaneously form an emulsion when mixed with gastric/intestinal fluid. Such emulsions, when formed, may provide for increased or improved stability of the fatty acids for uptake in the body and/or provide increased surface area for absorption. Further, the enteral fluid may be in the form of emulsions and formulations, e.g. where the active/nutritional ingredient is microencapsulated or in the form of a gel or semi-solid formulations. Preferably, the enteral fluid is in the form of a liquid or an emulsion.

If the prefilled dispenser is for delivery of pharmaceuticals these are preferably packed and administered independently of other nutrients.

The dispenser includes at least one flexible body portion providing a reservoir, i.e. an inner volume, for the enteral fluid. The flexible body portion of the dispenser is deformable by hand power. By squeezing or putting pressure on the body portion, the enteral fluid therein will be forced from the inner volume of the body portion and out through the outlet. Hence, unlike a syringe, the dispenser of the invention comprises a flexible body portion which comprises a wall material which itself is soft and compressible. Accordingly, when the dispenser outlet is attached to a feeding tube the flexible body portion of the dispenser can be gripped by a user's hands and be manually operated by putting pressure on the flexible body portion to press the enteral fluid through the outlet and into a feeding tube. The inner volume of the flexible body portion is e.g. 1 to 30 ml, more preferably 2 to 20 ml or 2 to 15 ml, and most preferably 2 to 8 ml. In a particularly preferred embodiment the dispenser has a total volume of about 3-6 ml.

Although the flexible body portion is deformable, it also provides some rigidness. Combined with a small volume size this rigidity makes the dispenser able to be held in an upright position when attaching the outlet to the feeding tube. Accordingly, there is preferably no means for hanging or holding the dispenser other than by attaching the outlet to the feeding tube, e.g. to a port of such. The flexible body portion of the dispenser is hold-able by one hand and the enteral fluid is dispenseable into the enteral tube by manually pressing the flexible body portion using the hands. Further, to keep the design simple, and as the size is so small, there is no need for any pressurizing means, energy source or venting means, and the dispenser preferably does not include such.

Attached to the flexible body portion one or more grip portions may be present, making it easy for the user to grip the dispenser, to attach it to the feeding tube or to hold the dispenser during use when pressing the content from the inner volume, through the outlet and into the feeding tube. The grip portion may comprise one or more flaps attached on one part of such to the flexible body portion. One example of such grip portion is a pier handle, such as a rounded handle extending from the surface of the body portion, e.g. in a distal relation to the outlet. Preferably, the grip portion comprises ridges or grooves at the surface of the grip portion, enabling a firm grip of this.

In some embodiments the flexible body portion has a pouch format or a tube format. In these embodiments, the flexible body portion comprises one or more sidewalls joined to a bottom portion, preferably wherein the bottom portion defines a base having geometry sufficient for enabling the sidewalls to remain generally upright relative to a horizontal surface onto which the base has been placed. The sidewalls, such as the sleeve of a tube, are joined to each other by either none, one or two seams. In one embodiment, the sidewalls are seamless and only joined at the bottom portion and top portion. The bottom portion, and also the horizontal cross-section, of the pouch or tube is preferably either elliptical or circular. Preferably, when the cross-section is circular the sidewalls are seamless or joined to each other by one seam and when the cross-section is elliptical the sidewalls are joined to each other by two seams. Preferably the outlet of the dispenser is placed at the bottom portion, preferably centrally positioned to the bottom, providing a bottom outlet. The sidewalls are further joined at a top portion, in a distal relation to the bottom portion. The cross-section area may vary from the bottom portion to the top portion. Accordingly, the flexible body portion is not restricted to having a classical pouch or tube shape, but may also take the shape of e.g. an hourglass or cone, having its base either up or down. In said embodiment a grip portion may be attached to the top portion. In one embodiment, the bottom portion is simply formed by the sidewalls meeting by the outlet. In another embodiment, the bottom portion has an angular geometry such as a rhombus, to cause less stress to the wall material. In a preferred embodiment, the flexible body portion has a tube format. More preferably, the tube includes a grip portion forming a top portion in a distal relation to the outlet.

In another embodiment the flexible body portion comprises two compartments, making a dual pouch format. A first pouch is preferably prefilled with the enteral fluid, while a second pouch is prefilled with a washing medium, such as saline or another water-soluble liquid with or without antimicrobial preservatives and with or without one or several surfactants to better wash away remnants of the enteral fluid. Alternatively, the second pouch is prefilled with another enteral fluid selected from the ones provided above. Yet, alternatively, the second pouch is prefilled with a lipase which upon mixture with the fatty acid or lipid will hydrolyse this. Hence, the two pouches may be prefilled with different enteral fluids selected from compositions of nutrients or pharmaceuticals, or any combinations thereof. For example, this may be done if the additional enteral fluid of the second pouch is not stabile or mixable with the enteral fluid of the first pouch. Each pouch comprises a pair of sidewalls joined to a bottom portion. The first and second pouches are separated from each other, providing two separate compartments, and are coupled to each other by the outlet. The pouches are preferably secured together at the bottom portion, e.g. at the bottom marginal edges or part of the sidewalls. Each pouch has an individual sealed opening in communication with the outlet of the dispenser, preferably at the bottom portion of each pouch. Preferably, the pouches are individually sealed by a weak seal which is ruptured by pressure resulting in emptying the pouches individually through the common outlet. The two pouches are arranged to enable the contents of the two compartments to be ejected, e.g. squeezed from the dispenser sequentially or simultaneously through the common outlet. In one embodiment the two pouches resemble two wings positioned away from a vertical axis running through the outlet. Each pouch optionally and individually comprises a grip portion attached thereto.

In yet another embodiment, the flexible body portion resembles a bellows type dispenser. In this embodiment at least part of the walls of the flexible body portion are foldable, such that when the dispenser is squeezed the walls fold together to reduce the inner volume (reservoir) and hence the content is pressed towards and throughout the outlet. The outlet of the dispenser is placed at a bottom portion of the flexible body portion, preferably centrally positioned, and preferably the outlet going generally perpendicular to the bottom portion. In one embodiment the flexible body portion comprises a pair of non-foldable sidewalls joined to each other by foldable walls. The non-foldable sidewalls are further joined to the bottom portion, preferably wherein the bottom portion defines a base, and the sidewalls are further joined at a top portion, in a distal relation to the bottom, and wherein the top portion is foldable. In this example, the flexible body portion is squeezable in the horizontal direction by pressing the non-foldable sidewalls together. In another example, the flexible body portion has a cylindrical shape, preferably with a circular base, wherein one base forms the bottom portion wherein the outlet is and the other base forms a top portion, and wherein the sidewall extending between the bottom portion and top portion is foldable. In this example, the flexible body portion is squeezable in the vertical direction by pressing the top portion and bottom portion towards each other. In one embodiment for the bellow type dispenser, the flexible body portion includes a clip or lock attached on one side to the flexible body portion adapted to hold two part which have been pressed towards each other, e.g. two side walls or the top portion and the bottom portion, together when the dispenser is emptied. The two parts will then remain in a joint position both to indicate the dispenser is used/empty and to prevent and avoid backflow of fluid into the reservoir of the flexible body portion. An advantage with this embodiment wherein the flexible body portion resembles a bellow type dispenser is that the designs allow for one-hand operation, for both connecting the dispenser to a feeding tube and for dispensing the fluid into this.

Preferably, the dispenser comprises a flexible body portion which is easy to empty by manual force, to enable the correct dose of enteral fluid to the user. Preferably, when having used the dispenser there is less than e.g. 5 vol % remaining enteral fluid in the dispenser, such as less than 4 vol %, 3 vol % or 2 vol %. The flexible body portion of the dispenser is deformable. By putting gentle pressure on the flexible body portion the enteral fluid therein will be forced through the outlet. The flexible body portion delivers the enteral fluid therein on demand without the required use of a pump or external energy source of any kind. In some embodiments of the invention one can simply squeeze the flexible body portion with the fingers to dispense the stored fluid through the outlet. In other alternatives, the walls or top and bottom of the flexible body portion are folded and can be pressed together with the forces of the fingers. In a preferred embodiment the dispenser can be operated with only one hand. In yet another alternative, some means for pressing the enteral fluid towards the outlet is attachable to the flexible body portion. Such pressure means may be re-usable. Said pressure means is either separate to the flexible body portion and attached when needed, or is an integrated part of the flexible body portion. Such pressure means may be selected from the group of clamps and sliders. In one example, the pressure means comprises a pull down slider. Such slider is adapted to embrace the flexible body portion, such as going across a pair of sidewalls of this. The slider may be openable, like a clamp, or may be closed unit, like a rubber band. The slider is movable such as between a top portion and a bottom portion of the flexible body portion, squeezing the sidewalls firmly together and thus pressing the enteral fluid towards the outlet. In another embodiment the pressure means comprises a clamp, such as two stiff walls joined together on one side, which may be attachable to the flexible body portion and used for pressing two walls together and pressing the enteral fluid towards the outlet. The slider/clamp could be for one time use or could be reusable and is of material such as plastic, metal or carton.

The outlet of the dispenser is arranged to the flexible body portion. Hence, the outlet is e.g. an integrated part of the flexible body portion. The outlet preferably has an outlet body extending in fluid communication between a first opening and a second opening thereof. Hence, preferably the outlet constitute a spout. The outlet is attached to, i.e. in fluid communication with, the flexible body portion providing a passageway from the reservoir of the flexible body portion and through the first opening, the outlet body and the second opening of the outlet. The flexible portion is accordingly united with the outlet. In one embodiment, the first opening of the outlet forms an integrated part of a bottom portion of the flexible body portion. Preferably, the passageway of the outlet body is positioned generally perpendicular to the bottom portion. The second opening is the outer opening.

The outlet of the dispenser is connectable with a feeding tube, preferably via a connection component such as a tube port, such as the feeding port or the medical port. When the outlet of the dispenser is attached to a port of a feeding tube by attaching any of the outlet body and the first or second opening of the outlet to the port, there is an interphase between the outlet and the feeding tube port and a passageway is provided extending in fluid communication between the reservoir of the flexible body portion of the dispenser and the inner volume of the feeding tube. The outlet of the dispenser is configured to be adapted to fit standardized tube feed entries, and the outlet can thus have different configurations. The outlet is configured so as there is interference fit between this and the port of the feeding tube. The outlet is e.g. configured to have e.g. Luer connection capability, Enlock connection capability or ENFit connection capability.

In one alternative embodiment the outlet of the dispenser provides one part of a Luer taper connection. The Luer taper is a standardized system of small-scale fluid fittings used for making leak-free connections between a male-taper fitting and its mating female part. There are two varieties of Luer taper connections: Luer Lock and Luer Slip. Luer Lock fittings are securely joined by means of complementary threads and tabs on the two parts, and the two parts are twisted and locked together. In the field of administration of nutrition Luer Lock systems have been reserved for parenteral nutrition, hence Luer Lock is currently not usable for dispensers for enteral nutrition. Luer Slip fittings, also called Slip fit systems, are pressed together and are held together by friction only, without any threads. The outlet of the dispenser may be configured such that it provides one part of a Luer taper connection either of the Luer Lock type or the Luer Slip type, or of both. The outlet of the dispenser is configured to function as either the male or the female entity of such connection. In this alternative, the outlet of the dispenser is preferably a Luer Slip part, and preferably the male part. Accordingly, in this embodiment the outlet typically comprises a tapered tip extending between first and second opening for inserting into a tube port providing a leak proof connection about the outlet. The outlet is made suitable for use with a Luer Slip connection component of a feeding tube.

As the standards for, and requirements to, feeding tubes and enteral delivering systems are changing also new delivery systems are needed. The International Standards Organization have created ISO CD 80.369-3 defining a safe design for enteral feeding connectors. Feeding tubes with new so called ENFit male connectors are introduced and the feeding tube port for administration sets are changing to male ports. Accordingly, and preferably, the outlet of the dispenser is configured to be according to the new standard, configured so there is an interference fit between the outlet of the dispenser and the ENFit port of the feeding tube, having complementary design. In this alternative the dispenser outlet preferably provides a female ENFit connector part which fits the new ENFit connector system, and this part can be connected with a male ENFit part connection, such as the tube port providing ENFit capability. The ENFit connector has a unique enteral-specific design that provides a locking feature ensuring appropriate connection. In a preferred embodiment, the invention provides a dispenser according to any of the above comprising an outlet with ENFit connectivity, preferably comprising a female ENFit connector end. Accordingly, in this embodiment the dispenser comprises an outlet with female ENFit lock, as corresponding syringes and feeding bag sets will have to have, matching the new male ENFit lock ports, instead of the traditional Y-ports. In a particularly preferred embodiment, the flexible body portion of the dispenser has a tube format and the outlet has ENFit connection capability, and most preferably the dispenser has a tube format and the outlet has a female ENFit lock.

When the outlet of the dispenser is attached to a port of a feeding tube by attaching the outlet body and/or the first or second opening of the outlet to the port a passageway is provided extending in fluid communication between the inner volume of the flexible body portion of the dispenser and the inner volume of the feeding tube. The passageway of the outlet is typically tubular or provided with conical geometry between the first and second opening. The outlet has an outlet body extending between the first and the second opening and this may have an outwardly-facing frustroconical surface, i.e. it is tapered, providing a spout. The inner passageway may be generally tubular or frustoconical, independently of the outwardly-facing geometry. The outlet body has a length that generally corresponds to the length of a neck of a tube port, depending on the configuration of the port.

The dispenser is ready for use, being prefilled with the enteral fluid and being easily attachable to a feeding tube. The prefilled dispenser provides a single dose, a so called single dose delivery package. The dispenser is non-refillable. Further, the dispenser is non-reusable. The content of the dispenser is easily squeezed directly into a feeding tube or a port of such throughout the outlet and can be supplied to a subject independently of any other enteral nutrition given to the subject. Accordingly, the enteral fluid of the prefilled dispenser is preferably not to be connected to other containers, such as bags, syringes or cartridges, but is provided directly to the subject in the tube or tube port. Alternatively, in one embodiment the device is connectable with other containers such as bags, syringes or cartridges. Further, the dispenser has only one opening and that is the outlet. Accordingly, there is no inlet to the dispenser or to the reservoir or inner volume of this. In another embodiment, the content of the prefilled dispenser of the invention may be administered directly orally. After use the dispenser can be discarded, and for the next supplement to be administered a new prefilled dispenser is to be used. Before being used, the outlet of the dispenser is closed with closure means, such as selected from the group of caps, seals, lids, tear-off parts, twist-away parts and spikes to be pushed in. In one embodiment the closure means include both an inner and an outer closure means, respectively closing the first and second opening of the outlet. In another embodiment, only one of the first and second openings is closed by a closure means. The first opening is preferably closed by a seal. Such seal may be a membrane or foil of wall material compatible with the enteral fluid, e.g. including the same material as an inner layer of the wall material used, alternatively the seal is a seam. In some embodiments the outlet is opened by squeezing the dispenser sides to break such closure seal, and the dispenser outlet is configured such that this preferably is to take place after any closure means closing the second opening has been removed and the dispenser has been attached to a feeding tube. The second opening is preferably closed by a cap, a tear-off part or a lid. Such closure means is configured to match the outlet configuration. The cap is e.g. selected from a pull-off or a twist-off cap, or a combination of such. A tear-off part is e.g. a tear-off plastic tip. Alternatively, the closure includes a twist away element, which can be twisted off, in particular along a predetermined breaking line. In one embodiment the closure means for the second opening is re-attachable. In another embodiment, the closure means is non re-attachable after it has been opened, e.g. when this comprises a twist away element. Dispensers having closure means which are re-attachable are favorable to avoid spilling of any remaining fluid after the dispensers have been used, by putting the closure back on the dispenser. In one embodiment the closure includes a tamper-evident feature showing whether the closure has been removed or tampered with.

The dispenser, comprising the flexible body portion and the outlet, comprises a wall material which is compatible with the prefilled fluid therein and which protects this. The wall material of the dispenser further ensures a long shelf stability of the enteral fluid in the dispenser. Using the dispenser of the invention, the enteral fluid in this has a shelf life of at least 1 year, preferably at least 2 years and most preferably at least 3 years. The compatible wall material is used for surfaces of the dispenser being in direct contact with the enteral fluid prefilled in the dispenser. Preferably the wall material is used in the flexible body portion and particularly in either of the walls, top and bottom portion of this. The wall material is preferably the material that constitute the reservoir for the enteral fluid in the flexible body portion. In one embodiment, the wall material protects the enteral fluid from oxidation. In a further embodiment, the wall material comprises a multibarrier layer providing protection for the enteral fluid prefilled in the dispenser. In this embodiment, the wall material comprises two or more main layers, such as three to seven layers, and in a preferred embodiment the wall material comprises three to five main layers. In addition to the main layers which all preferably have a function, optional adhesive layers may be present between these. Such multibarrier layer may comprise an inner layer which is compatible with the enteral fluid and prevents interaction with the enteral fluid prefilled in the dispenser, another layer which provides a barrier against oxidation and an outer layer which provides mechanical strength to the dispenser. In one alternative at least one layer comprises polymers and one layer comprises inorganic material. Preferably the inner layer is a layer being in contact with the fluid in the dispenser, the another layer is an intermediate layer between the inner and outer layer, and outer layer is preferably a visible layer. The inner layer, preventing interaction with the fluid of the dispenser, is made of a material which is compatible with, and which does not react with, the enteral fluid, such as with a composition of fatty acids or lipids. With an appropriate wall material there is no chemical reaction between the wall material and the enteral fluid such as migration of fluid components into wall, no migration of wall material/plastic components or layer adhesives into the enteral fluid, and no chemical reactions between wall and fluid components at the surface of the wall. In one alternative such inner layer comprises polyolefins, more preferably polyethylenes (PE) or polypropylenes (PP), such as high density polyethylene (HDPE) or low density polyethylene (LDPE). For enteral fluids comprising compositions of fatty acids or lipids and lipid-based formulations, such as omega-3 fatty acids and derivatives thereof, the group of polyolefins such as: LDPE, HDPE, PE and PP, are identified as the best candidates for direct contact with the fluid, as shown in Example 1. The inner layer of the wall material may be in the form of a thin film, such as a stretch or cling film. For the intermediate layer providing a barrier against oxidation, such as oxidation of fatty acids or lipids filled in the dispenser, a material commonly used as oxygen barrier in food packing may be used. Such materials include polymers, such as ethylene vinyl alcohol (EVOH), and foils such as aluminum oxide or silicon oxide. For oxygen sensitive enteral fluids such as fatty acids and lipids, a multilayer structure with an oxygen barrier material comprising e.g. an EVOH polymer, silicon oxides (SiOx) or aluminum foil is required in order to achieve the necessary oxygen barrier property. The layer providing mechanical properties to the wall material, preferably comprises a polymer, e.g. polyolefins like LDPE, HDPE, PE and PP or polyethyleneterephthalate (PET) or polyamides (PA). PET or PA could also be added as potential additional outer layers. In addition to the mentioned layers adhesives can be included between these, such as different adhesive resins also called tie-layers. In one alternative the wall material comprises an oxygen barrier, such as an EVOH layer, sandwiched between two polyolefin layers, such as between polyethylene and/or polypropylene layers. In one alternative, an aluminum-based oxygen barrier layer is sandwiched between polyolefin layers, such as between polyethylene layers. In a preferred embodiment the wall material of the dispenser is transparent. Preferably the dispenser is in a pouch or tube format having transparent wall material. One advantage with this is that the content of the dispenser can be seen by the user and based on the appearance of the content confusion with other products may be avoided. Further, the wall material of the dispenser is also optionally moisture proof and/or impermable to microorganisms. In one embodiment, the wall material comprises only one material, i.e. only one layer, which provides the appropriate properties for being compatible with the enteral fluid, and also preferably protecting this as described above.

To keep the flexible nature of the plastic, the total layer thickness of the wall material should be kept as thin as possible, preferably below 2000 μm. More preferably, the wall material thickness is below 1000 μm and most preferably about 500 μm or below. The individual layer thicknesses may vary. The oxygen barrier layer could be in the range 1-25% of the total thickness, preferably between 2-10%, depending e.g. on the material chosen.

Table 1 below provides three examples of different multilayers which may be used in the wall material of the dispenser of the invention. Combinations of these may also be used, such as selecting one layer from one example and another layer from one of the other examples. In Table 1 the outer layer is numbered 1 and layer 5 or 6 is the inner layer which is in contact with the enteral fluid. The inner layer may also be thinner than those included in table 1, such as having a thickness in the range of about 10-150 μm. The outer layer may also be thinner than those included in table 1, such as having a thickness in the range of about 75-400 μm.

TABLE 1

| Layer | Thickness (μm) | Material type |
|---|---|---|
| 1 | 320 | PE-LD Colorant |
| 2 | 38 | Colorant Copolymer |
| 3 | 40 | Aluminium |
| 4 | 38 | Copolymer |
| 5 | 63 | PE-HD |
| 1 | 345 | PP |
| 2 | 30 | PP |
| 3 | 5 | Adhesive |
| 4 | 15 | Aluminium |
| 5 | 5 | Adhesive |
| 6 | 100 | PP |
| 1 | 150 | PP |
| 2 | 40 | PP |
| 3 | 8 | Tie-layer |
| 4 | 12 | PET/SiOx |
| 5 | 8 | Tie-layer |
| 6 | 80 | PP |

In a preferred embodiment the dispenser is a prefilled dispenser providing a single dose delivery package of a supplemental enteral fluid. The dispenser as disclosed may be usable with different types of enteral fluids, but in a particularly preferred embodiment the dispenser is prefilled with a fatty acid oil mixture comprising long chain polyunsaturated (LC-PUFA) fatty acids, preferably comprising omega-3 fatty acids, providing a small-volume dispenser which easily can be coupled to a tube port providing the relevant dose of fatty acids to a subject in need of enteral nutrition. Accordingly, the dispenser is configured to contain a maximum total fill weight of about 30 grams, preferably maximum 25 grams, and most preferably maximum 20 grams. The total omega-3 fatty acid weight in the fluid prefilled in the dispenser is e.g. up to 20 grams, more preferably up to 5 grams providing an adequate single dose, and most preferably the dispenser comprises 1-5 grams of omega-3 fatty acids. Further, in a particularly preferred embodiment the dispenser has a flexible body portion of a tube format and the outlet of the dispenser has ENFit connection capability. Further, such dispenser is preferably prefilled with a fatty acid oil mixture comprising long chain polyunsaturated (LC-PUFA) omega-3 fatty acids.

In a second aspect the invention provides a method for administering an enteral fluid to a subject using the dispenser of the first aspect. Accordingly, the method comprises the steps of attaching a dispenser prefilled with an enteral fluid to a port of a feeding tube, wherein the dispenser comprises i) a flexible body portion providing a reservoir for the enteral fluid, the flexible body portion comprising a wall material compatible with the enteral fluid, wherein the flexible body portion having arranged thereto; ii) an outlet connectable with a feeding tube; and putting gentle pressure on the flexible body portion to press the enteral fluid though out the outlet. The enteral fluid is hence provided to a subject taking enteral nutrition and who is in need of or wants a supplemental enteral fluid, such as a composition of fatty acids or lipids. In this method the dispenser is attached to a port of a feeding tube by firstly optionally removing a closure means closing the outlet of the dispenser. In one alternative the dispenser is opened simply by connecting the dispenser outlet to the tube port and squeezing the flexible body portion of the dispenser, optionally to remove a closure means such as breaking a closure seal closing the outlet. If the dispenser includes a closure means which must be manually removed, such as by tearing off a seal or removing a cap, this should be done shortly before attaching the dispenser to the tube port. The dispenser is further attached to the port with a matching configuration, such as by providing a Luer taper connection or an ENFit connection. For example, if the outlet is configured as a Luer Slip male part this is inserted into a tube port configured as the corresponding Luer slip female part, and these are pressed together. When the dispenser is attached to the port of the feeding tube the enteral fluid can be pressed out of the dispenser and into the tube. By the method of the invention the enteral fluid in the prefilled dispenser is delivered independently of any other enteral nutrition administered to the subject, such as from a package being a bag, bottle or syringe which is connected to the same feeding tube as the one used in the method. In one embodiment, the dispenser of the invention is coupled to the same port, i.e. the feeding port, normally used for the enteral nutrition. In this embodiment, the enteral feed is provided before or after the enteral nutrition has been provided, and when the package for this is not coupled to the tube. In another embodiment, the dispenser of the invention is coupled to another port than the one used for enteral nutrition. In this embodiment of the method, the enteral fluid is administered before, after or simultaneously with the enteral nutrition.

When the flexible body portion comprises two compartments, making a dual pouch format, the method of administering the enteral fluid to a subject includes the step of putting pressure on the flexible body portion to press the enteral fluid through-out the outlet, wherein the first pouch containing the enteral fluid is preferably squeezed first followed by squeezing the second pouch containing a washing medium, or alternatively a second enteral fluid, to rinse out any residual content of the enteral fluid remaining in the bottom portion of the flexible body portion or in the outlet or tubing. In yet an alternative, the content of the two compartments are mixed prior to or when entering into the outlet. In this second aspect the same embodiments apply regarding the dispenser and the enteral fluid as for the first aspect.

In a third aspect, the invention provides a method for supplementing an enteral nutritional product with an enteral fluid, the method comprising the steps of attaching a dispenser prefilled with the enteral fluid to a port of a feeding tube, wherein the dispenser comprises i) a flexible body portion providing a reservoir for the enteral fluid, the flexible body portion comprising a wall material compatible with the enteral fluid, wherein the flexible body portion having arranged thereto; ii) an outlet connectable with a feeding tube; and putting pressure on the flexible body portion to press the enteral fluid through-out the outlet and into the feeding tube and; separately or simultaneously dispensing the enteral nutritional product into the same feeding tube. The enteral nutritional product is typically chosen among the standard enteral nutrition diets. In this third aspect the same embodiments apply regarding the dispenser and the enteral fluid as for the first aspect. In this embodiment, the invention provides a method for supplementing an enteral nutritional product with an enteral fluid comprising nutrients and pharmaceuticals, wherein the nutrient may be selected from the group of Enteral formulas for Special Medical use, Foods for Specified Health Uses, Food for Special Medical Purposes (FSMP), Food for Special Dietary Use (FSDU), medical nutrition, medical food, clinical nutrition, nutraceuticals and supplements.

In a further aspect the invention provides the use of a dispenser as disclosed in the first aspect for delivery of an enteral fluid, provided as tube feed. In another aspect the invention provides the use of the dispenser as disclosed in the first aspect for supplementing an enteral nutritional product with an enteral fluid as disclosed. Further, the invention provides the dispenser as for delivery of an enteral fluid or for supplementing an enteral nutritional product.

Different techniques can be used when producing the dispenser of the invention. One option is so called blow fill seal (BFS) wherein a container, such as the dispenser of the invention, is formed, filled, and sealed in a continuous process without human intervention, in a sterile enclosed area inside a machine. Reference is made to standard known processes. A second option is preparation of a tube using an extrusion process. A sleeve may then first be produced from a specific laminate to make up the wall material. Once the sleeve, preferably seamless, is produced the tube head, i.e. the bottom portion and outlet, is fitted using an automated heading machine. The open tubes are then most commonly filled through the top portion, opposite the outlet, and then crimped to be closed. A third option is producing a pouch from two layers of laminate, attaching an outlet, and sealing these together using heat.

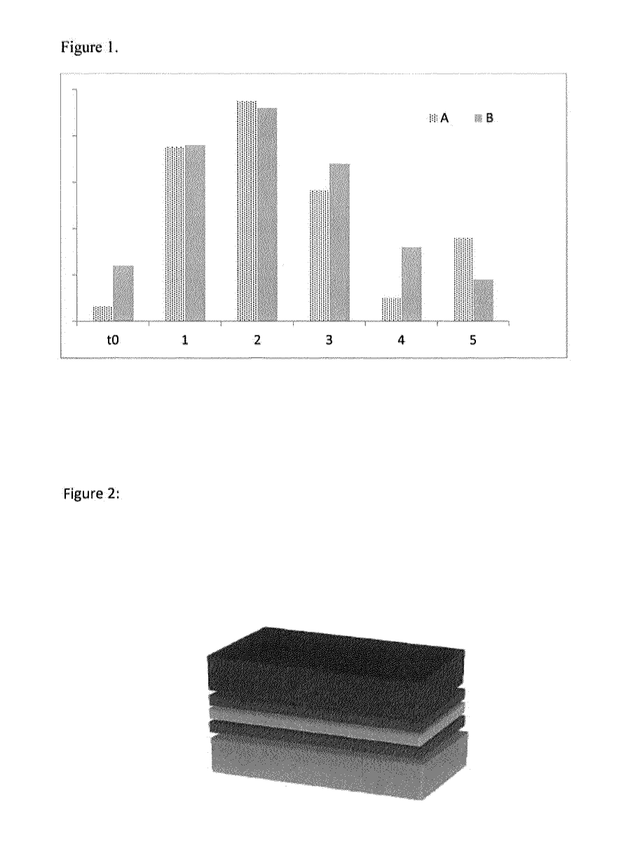

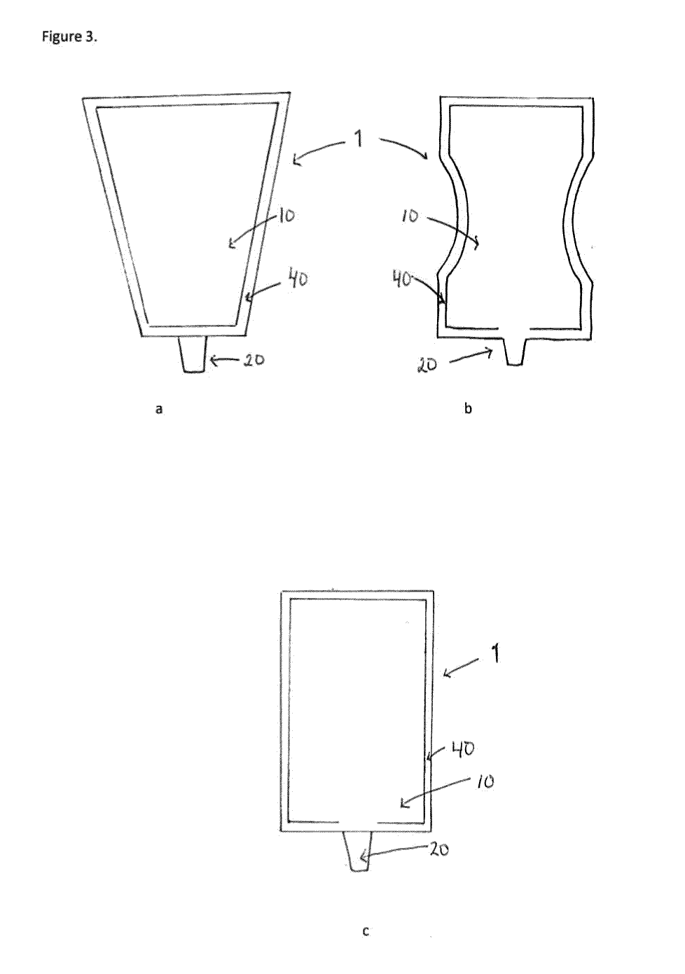

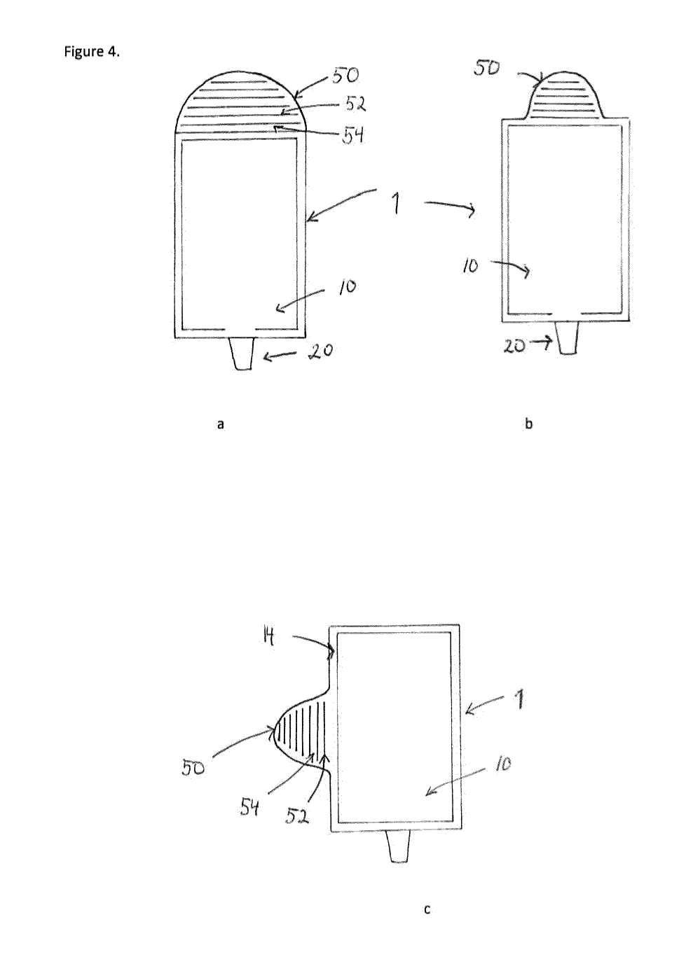

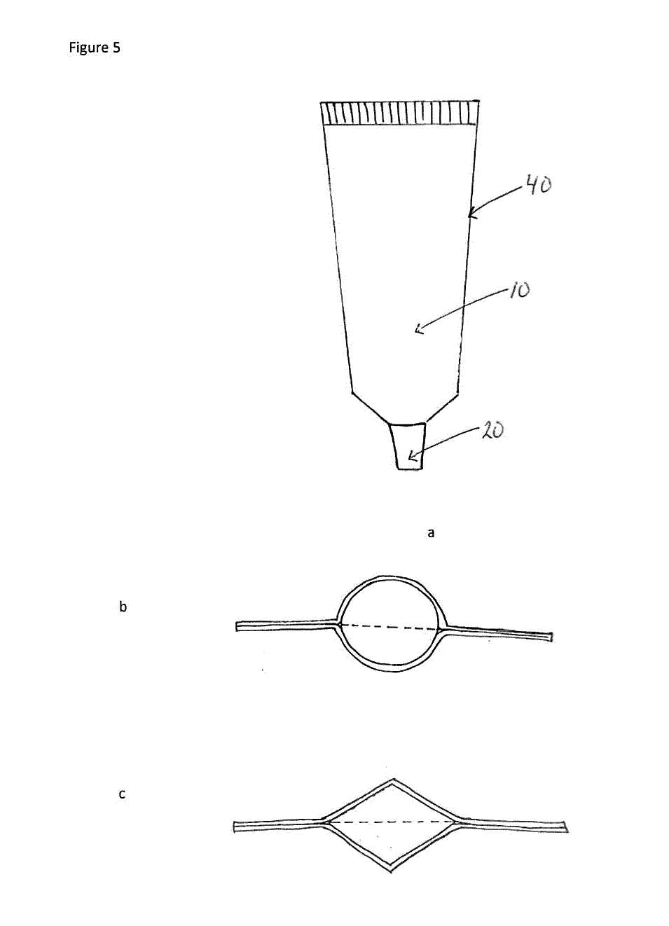

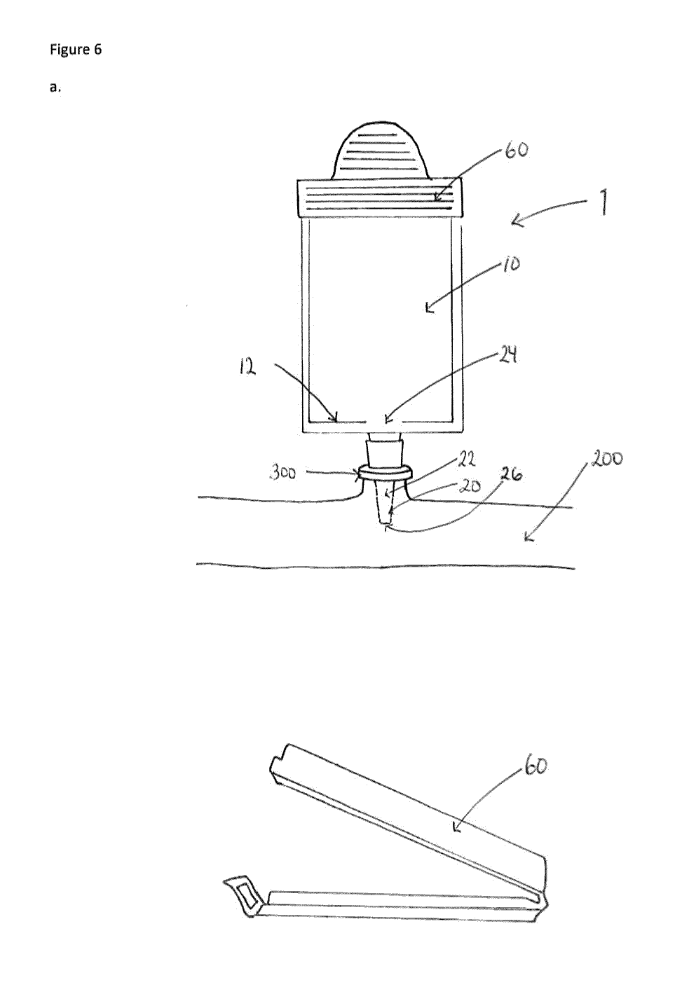

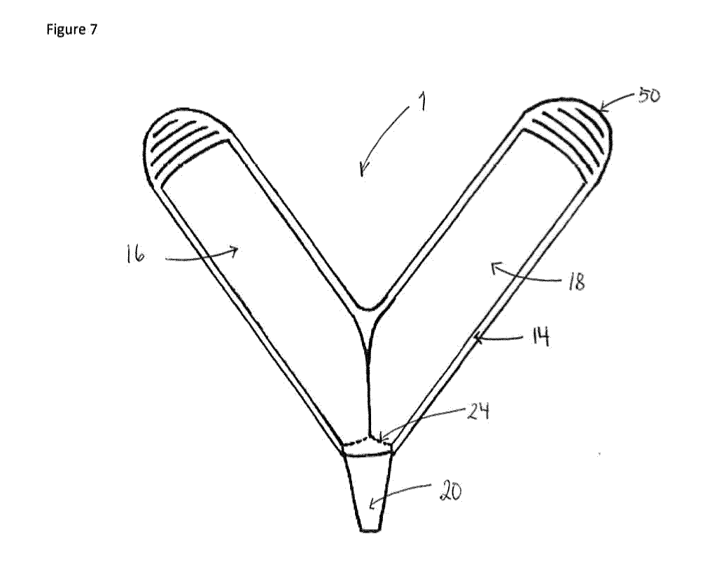

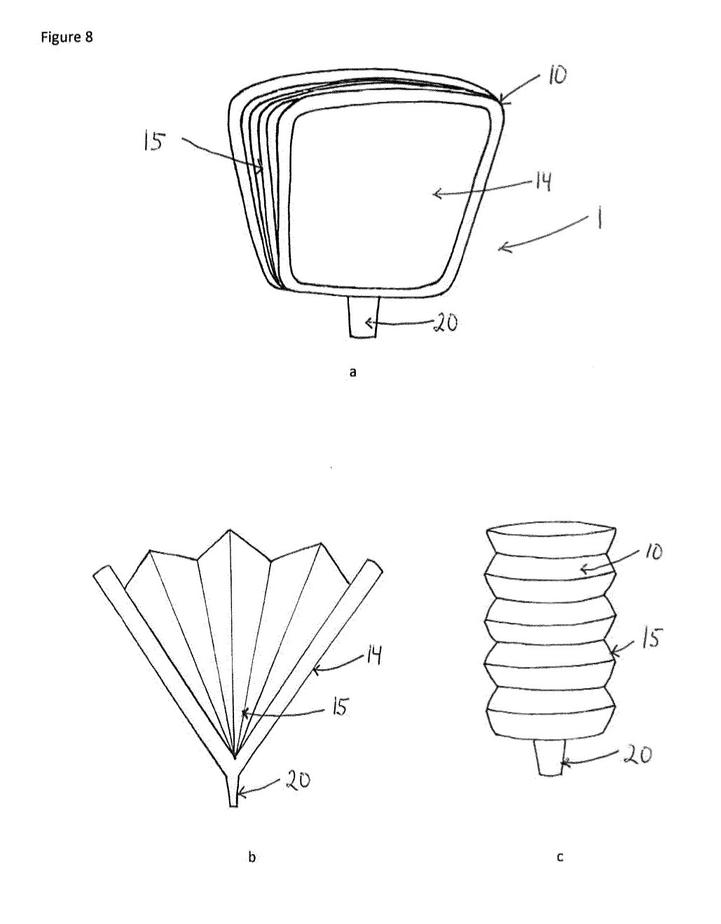

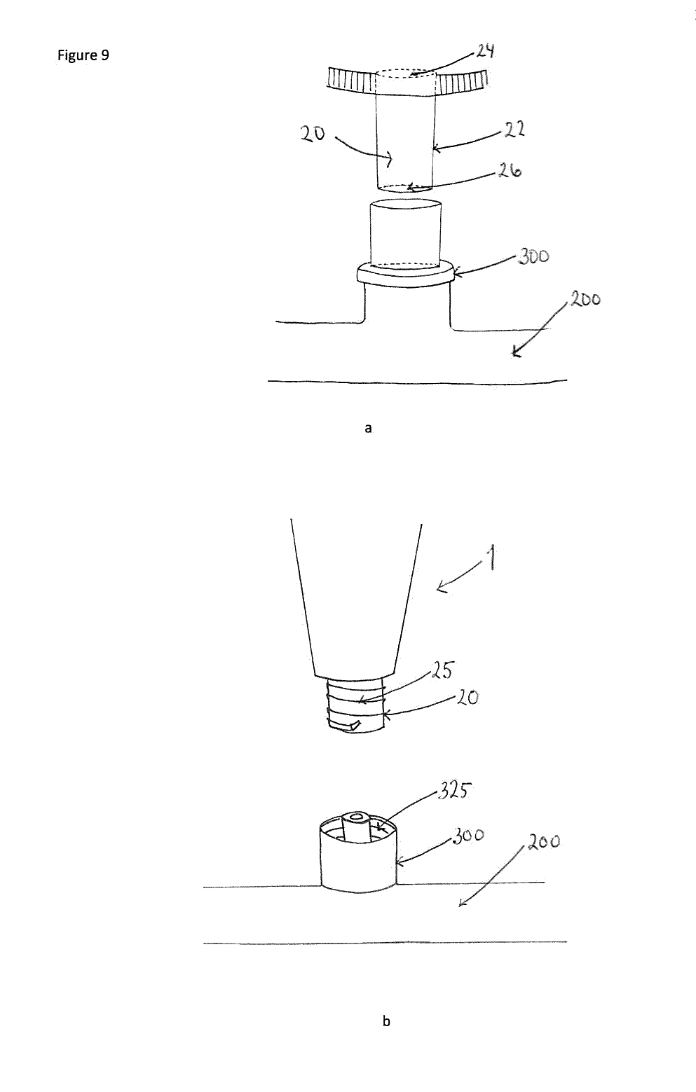

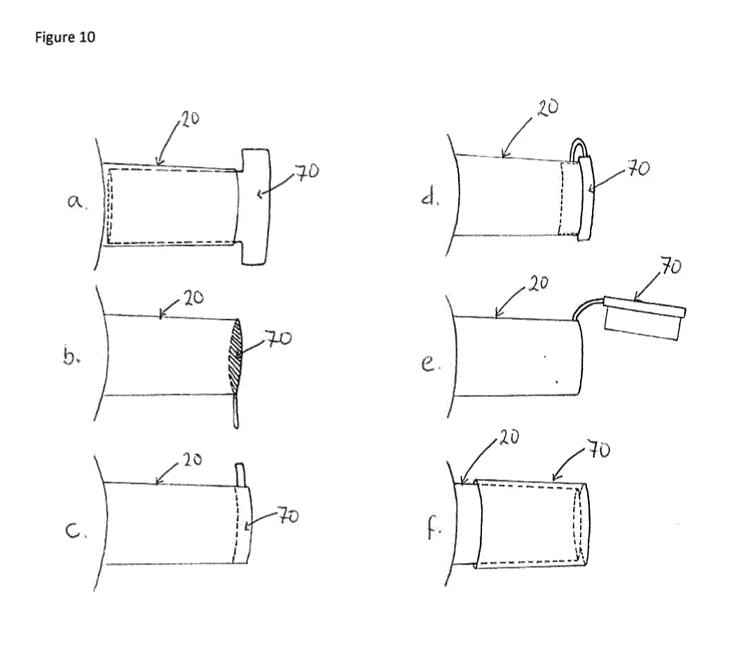

Preferred embodiments of the invention will now be described by way of example only and with reference to the accompanying drawings, in which FIG. 1 is related to Example 1 providing the oxidation parameter results from the trial of Example 1. FIG. 2 provides a sketch of a multibarrier layer which was used in Example 1 as one of the test wall materials, consisting of a three-layer structure of LDPE/EVOH/LDPE, with adhesion layers between the main layers. Further, FIGS. 3a-c provide sketches of alternatives of a dispenser according to the invention, seen from the outside, wherein this has a pouch format, wherein the dispenser 1 comprises a flexible body portion 10, with a wall material 40, having arranged thereto an outlet 20. FIGS. 4a-c provide the dispenser 1 further comprising a grip portion 50, making it easy for the user to grip the dispenser. In FIGS. 4a and 4b the grip portion 50 is attached to the flexible body portion 10 at the top portion in a distal relation to the outlet 20, wherein the grip portion 50 has ridges 52 and groves 54. In FIG. 4c the grip portion 50 is attached to the flexible body portion 10 at one sidewall 14. FIG. 5a provides a sketch of the dispenser in a tube format wherein the flexible body portion 10 has wall material 40 and an outlet 20. FIGS. 5b and Sc show cross-sections of alternative dispensers, having a flexible body portion having a circular cross-section, FIG. 5b, and a square cross-section, 5c. FIG. 6a shows one alternative of the dispenser 1 attached to a tubing 200, wherein the outlet body 22 of the outlet 20 has been inserted into a port 300 of the tubing 200, wherein the first opening 24 of the outlet 20 is part of the bottom portion 12 of the flexible body portion 10 and the second opening 26 of the outlet 20 is inside the tubing 200. FIG. 6a further shows that a pressure means 60, in the form of a slider, is attached to the flexible body portion 10, which can be slided down towards the outlet 20 to press the content of the dispenser out of the dispenser and into the tubing 200. FIG. 6b shows one example of a pressure means 60, which is attachable to the dispenser 1, as shown in FIG. 6a. In FIG. 7 a dispenser 1 of the invention is shown, wherein the flexible body portion comprises two compartments; a first pouch 16 and a second pouch 18 each comprising sidewalls 14 with a wall material as disclosed, wherein the pouches, in this example, each comprises a gripping portion 50 attached to pouches 16 and 18 in distal positions to the outlet 20. Each pouch has a first sealed opening 24, which is designed to rupture when the pouch is squeezed. FIGS. 8a-c provide dispensers of the invention of the bellow-type. FIGS. 8a and b show one version of a bellow-type dispenser in front view and side view respectively, wherein the flexible body portion 10 comprises non-foldable sidewalls 14 which are joined to each other by foldable walls 15. When pressing the non-foldable sidewalls towards each other, i.e. in the horizontal direction, the content of dispenser will be pressed towards the outlet 20. FIG. 8c shows an alternative bellow-type dispenser, wherein the flexible body portion 10 has foldable sidewalls 15, which are squeezable in the vertical direction towards the outlet 20. FIG. 9 shows how the dispenser of the invention is coupled to a port of a tube. FIG. 9a provides one alternative outlet 20, such as of the Slip fit type, which comprises an outlet body 22 extending between a first opening 24 and a second 10 opening 26. The outlet 20 is connectable to the tube 200 by inserting this into the port 300, matching the configuration of the outlet 20. FIG. 9b provides an alternative coupling which can be used between the outlet of the dispenser and the port showing the ENFit configuration for providing a secure fit, wherein the upper part of the figure shows a part of the dispenser 1 including an outlet 20 with a configuration of a female ENFit part comprising threads 25. The outlet 20 is connectable to the tube 200 by inserting and twisting this into the port 300 having a male ENFit configuration with threads 325, matching the outlet 20 of the dispenser 1. FIGS. 10a-f provide different types of closure means for the second opening of the outlet, for each figure the closure means 70 is attached to the outlet 20. FIGS. 10d and 10e show an outlet with the same closure means in closed and open position, respectively.

### EXAMPLES

### Example 1: Compatibility Trial with High Concentrated Fish Oil

Four different ampoule materials were filled with concentrated fish oil (PronovaPure 400:200 TG) comprising the omega-3 fatty acids EPA and DHA in triglyceride form, and placed at 40° C./75% relative humidity. The materials of the ampoules were:
1. Polypropylene
2. Low Density Polypropylene
3. High Density Polypropylene
4. Coextrusion—a multilayer laminate
5. Glass (as reference)

The results are provided in FIG. 1 wherein the t0 column shows the oil oxidation parameters at start (t0) and the other columns provide the oxidation parameters, i.e. the peroxidine value (A) and the anisidine value (B), for the oil after 2 weeks storage (at accelerated conditions, 40° C./75% Relative Humidity) in different materials. Semi-inert filling of ampoules resulted in high start oxidation values. The white/dotted columns show the peroxide value (A) a measure for primary oxidation, and the gray columns show the anisidine value (B), a measure for the secondary oxidation. The material no. 4, i.e. the multilayer laminate, consists of a three layer structure of LDPE/EVOH/LDPE, plus adhesion layers between, as shown in FIG. 2.

Conclusion: As can be seen from FIG. 1, the oxidation level for the fish oil was considerably lower for the fish oil stored in the ampoule of the multilayer laminate (material no. 4) than for the other materials. In this compatibility test it was confirmed that a multilayer laminate as the one used for material no. 4, comprising an oxygen barrier, is needed to avoid oxidation of the fish oil. Other materials comprising an oxygen barrier, such as one comprising aluminium or SiOx could also likely be used with similar result.

### Example 2: Tube Dispenser Filed with Omega-3 Fatty Acids

A tube dispenser according to the invention is prepared and filled with a fatty acid mixture for delivery to a subject enterally. The tube sleeve comprises a multilayer wall material with a total thickness of about 300 μm comprising an intermediate aluminum layer between polyethylene layers. A tube head, i.e. the bottom portion and outlet, is fitted to the seamless sleeve using an automated heading machine. The outlet is designed with a female ENFit connector part as shown in FIG. 9b comprising closure means. The tube is filled with an enteral fluid through the top portion, opposite the outlet, and crimped to be closed. The total volume of the dispenser is about 4 ml. The enteral fluid is selected from the following fatty acid mixtures comprising:
a) 360 mg EPA and 240 mg DHA per gram oil
b) 500 mg EPA and 200 mg DHA per gram oil
c) 460 mg EPA and 380 mg DHA per gram oil
d) 150 mg EPA and 500 mg DHA per gram oil
e) 150 mg EPA and 500 mg DHA per gram oil and lipid soluble vitamin D.
f) Emulsion containing omega-3 PUFAs in an amount of 75-200 mg/ml, providing a total volume of about 12 ml (Hence, the total volume of the dispenser is bigger for this).

The enteral fluid could also include other nutrients and pharmaceuticals according to the invention. The dispenser provides a single dose of about 3 grams omega-3 fatty acids, for example in the form of triglycerides. The dispenser is to be coupled to a tube port of a feeding tube wherein the port has a male ENFit connector part. The two parts are coupled by matching the complementary threads. The enteral fluid can then be administered. The dispenser is to be gripped by a user's hands and is manually operated by putting pressure on the flexible body portion of the tube to press the enteral fluid through the outlet and into the feeding tube.

### Example 3: Pouch Dispenser Filed with Omega-3 Fatty Acids

A pouch dispenser according to the invention is prepared and filled with a fatty acid mixture for delivery to a subject enterally. The flexible body portion of the pouch comprises a multilayer wall material with a total thickness of about 500 μm comprising an intermediate EVOH layer between polypropylene layers. A pouch spout, i.e. the bottom portion and outlet, is fitted to the pouch body between two side walls of the multilayer wall material and sealing these together using heat.

In this example the outlet is of the mail Slip fit type as shown in FIG. 9a comprising closure means.

The pouch is filled with an enteral fluid through the top portion, opposite the outlet, and crimped to be closed. Alternatively, the pouch dispenser is formed, filled and sealed in accordance with other standard available processes. The total volume of the dispenser is about 8 ml. The enteral fluid is selected from the following compositions comprising:
a) A preconcentrate comprising a fatty acid oil mixture comprising 500 mg EPA and 200 mg DHA per gram oil, and Polysorbate 20.
b) A fatty acid oil mixture comprising at least 90 percent EPA.
c) A fatty acid oil mixture comprising 60% by weight or more of EPA and DHA, and a-tocopherol (vitamin E).

The enteral fluid could also include other nutrients and pharmaceuticals according to the invention. The dispenser provides a single dose of about 3-5 grams omega-3 fatty acids. The dispenser is to be coupled to a tube port of a feeding tube wherein the port has a corresponding female Slip fit connector part. The two parts are coupled by pressing the outlet of the dispenser into the tube port. The enteral fluid can then be administered. The dispenser is to be gripped by a user's hands and is manually operated by putting pressure on the flexible body portion of the pouch to press the enteral fluid through the outlet and into the feeding tube.

1. A handheld dispenser for an enteral fluid, wherein the dispenser comprises
i) a flexible body portion providing a reservoir for the enteral fluid, the flexible body portion comprising a wall material compatible with the enteral fluid, wherein the flexible body portion having arranged thereto; and
ii) an outlet connectable with a feeding tube.

2. The dispenser of claim 1, wherein the dispenser is prefilled with an enteral fluid selected from nutrients and pharmaceuticals.

3. The dispenser of claim 2, wherein the nutrients are selected from compositions comprising fatty acids and lipids and derivatives thereof.

4. The dispenser of claim 3, wherein the fatty acids comprise long chain polyunsaturated (LC-PUFA) omega-3 fatty acids.

5. The dispenser of claim 4, wherein the omega-3 fatty acids comprise either eicosapentaenoic acid (EPA) or docosahexaenoic acid (DHA), or a mixture of such.

6. The dispenser of claim 3, wherein the fatty acids or lipids are in a formulated form.

7. The dispenser of claim 1, wherein the dispenser is for one-time use.

8. The dispenser of claim 1, wherein the dispenser has a volume of 1 to 30 ml.

9. The dispenser of claim 1, wherein the flexible body portion has a pouch format or a tube format.

10. The dispenser of claim 1, wherein the flexible body portion comprises two compartments.

11. The dispenser of claim 10, wherein a first compartment of the flexible body portion is prefilled with an enteral fluid selected from nutrients and pharmaceuticals and a second compartment is prefilled with a washing medium, a second enteral fluid different from the enteral fluid of the first compartment, or with a lipase.

12. The dispenser of claim 1, wherein the flexible body portion resembles a bellows type bottle.

13. The dispenser of claim 1, wherein the outlet is configured to have a Luer connection capability or an EN Fit connection capability.

14. The dispenser of claim 1, wherein the wall material of the dispenser comprises a multibarrier layer.

15. The dispenser of claim 1, wherein the wall material protects the enteral fluid from oxidation.

16. The dispenser of claim 1, wherein the dispenser is operable by one hand.

17. The dispenser of claim 1, wherein the flexible body portion has a tube format.

18. The dispenser of claim 17, wherein the outlet has ENFit connection capability.

19. A method for administering an enteral fluid to a subject comprising the steps of
attaching a dispenser prefilled with an enteral fluid to a port of a feeding tube, wherein the dispenser comprises
i) a flexible body portion providing a reservoir for the enteral fluid, the flexible body portion comprising a wall material compatible with the enteral fluid, wherein the flexible body portion having arranged thereto; and
ii) an outlet connectable with a feeding tube; and
putting pressure on the flexible body portion of the dispenser to press the enteral fluid though out the outlet, and into the feeding tube.

20. A method for supplementing an enteral nutritional product with an enteral fluid, the method comprising the steps of
attaching a dispenser prefilled with the enteral fluid to a port of a feeding tube, wherein the dispenser comprises
i) a flexible body portion providing a reservoir for the enteral fluid, the flexible body portion comprising a wall material compatible with the enteral fluid, wherein the flexible body portion having arranged thereto; and
ii) an outlet connectable with a feeding tube; and
putting pressure on the flexible body portion of the dispenser to press the enteral fluid though out the outlet and into the feeding tube; and
separately or simultaneously dispensing the enteral nutritional product into the same feeding tube.
